(19) United States
(12) Patent Application Publication
(10) Pub. No.: US 2015/0238425 A1
(43) Pub. Date: Aug. 27, 2015

(54) MINI-TABLETS

(71) Applicant: DSM SINOCHEM PHARMACEUTICALS NETHERLANDS B.V., Delft (NL)

(72) Inventors: Gabriel Marinus Henricus Meesters, Echt (NL); Ferdinand Antoine Spros, Echt (NL)

(21) Appl. No.: 14/420,966

(22) PCT Filed: Aug. 26, 2013
(86) PCT No.: PCT/EP2013/067599

§ 371 Date: Feb. 11, 2015

(30) Foreign Application Priority Data
Aug. 28, 2012 (EP) 12182026.0

(51) Int. Cl. A61K 9/20; A61K 31/424; A61K 31/43

(52) U.S. Cl. CPC A61K 9/2077; A61K 31/43; A61K 31/424; A61K 9/2095

(57) ABSTRACT

The present invention relates to a composition comprising a first granule with an antibiotic and a second granule with a β-lactamase inhibitor wherein at least one of the granules is a mini-tablet (a particle with defined dimensions), a composition contained in a sachet, pharmaceutical compositions comprising mini-tablets, the use of mini-tablets and a process for the preparation of mini-tablets.

### FIELD OF THE INVENTION

The present invention relates to a composition comprising a first granule with an antibiotic and a second granule with a β-lactamase inhibitor wherein at least one of the granules is a mini-tablet (a particle with defined dimensions), a composition contained in a sachet, pharmaceutical compositions comprising mini-tablets, the use of mini-tablets and a process for the preparation of mini-tablets.

### BACKGROUND OF THE INVENTION

A tablet is a pharmaceutical dosage form comprising active ingredients and excipients, usually in powder form, pressed or compacted from a powder into a solid dose. The excipients can include diluents, binders or granulating agents, glidants (flow aids) and lubricants to ensure efficient tableting; disintegrants to promote tablet break-up in the digestive tract; sweeteners or flavors to enhance taste; and pigments to make the tablets visually attractive. A polymer coating is often applied to make the tablet smoother and easier to swallow, to control the release rate of the active ingredient, to make it more resistant to the environment (extending its shelf life), or to enhance the tablet's appearance. A tablet can be formulated to deliver an accurate dosage to a specific site and is usually taken orally.

For most antibiotic drugs such as β-lactam antibiotics, the size of a tablet ranges from approximately 5 to 15 millimeters. The upper end of this range is normally used and due to the fact that the amount of active substance to be administered is relatively high. This can be several hundreds of milligrams to a gram. Amoxicillin for example, one of the most prescribed antibiotic drugs, is formulated in tablets comprising 500 mg or 875 mg of the active ingredient amoxicillin trihydrate. The amounts of active ingredients can be even higher in combination tablets such as those wherein amoxicillin is combined with the β-lactamase inhibitor clavulanic acid. These combination tablets can contain, next to the amounts of amoxicillin trihydrate mentioned above, also clavulanic acid in amounts like 125 mg. Typically ratio's of amoxicillin trihydrate to clavulanic acid can be 1:1, 5:1, 6:1, 10:1, 11:1, 15:1, 16:1 or 20:1.

A major drawback of antibiotic drugs formulated as tablet is that administration is difficult for many patients. Notably children, the elderly, people in care centers and handicapped people often have difficulties swallowing these relatively large tablets. Hence there is a need for antibiotic drug formulations that can be administered orally without the drawback mentioned above.

### DETAILED DESCRIPTION OF THE INVENTION

The use of small tablets is known from the prior art. For example, A. E. Clausen et al. (J. Control. Release (2001) 75, 93-102) and GB 2176999 disclose a (controlled release) tablet with a diameter of 5 mm comprising an antibiotic. JP 54126722 discloses small-sized tablets of 5-8 mm in diameter comprising amoxicillin. EP 2420226, US 2004/096496, US 2012/027855 and WO 2007/106957 disclose (controlled-release) formulations of several drugs. Although the possibility of formulating into a mini-tablet is mentioned in these documents, no further detail (for instance as to the size of such a mini-tablet) is given. U.S. Pat. No. 5,900,252 discloses mini-tablets with a diameter of less than 5 mm comprising a single drug, which may be formulated into capsules, tablets, sachets or suspensions. US 2006/003005 discloses a tablet comprising, amongst others, an enteric coated mini-tablet of undefined size comprising one or more active ingredients. US 2012/003312 discloses multi-layer mini-tablets comprising one or more drugs. The mini-tablets may be encapsulated into a capsule. WO 02/49618 discloses pellets, formally not tablets, with a diameter of 1-5 mm and a length of 1-10 mm comprising an antibiotic. WO 2005/099672 discloses the combination of an antibiotic and a β-lactamase inhibitor in a single tablet comprising separate immediate- and slow release phases. WO 2006/110807 discloses a controlled release capsule comprising a single cephalosporin in two different mini-tablets of undefined size, one coated, another un-coated. WO 2007/059916 discloses a small tablet of undefined size comprising an antibiotic. The tablet is coated with a film comprising a β-lactamase inhibitor. WO 2007/110875 discloses mini-tablets of undefined size comprising amoxicillin filled into gelatin capsules. WO 2008/010784 discloses nanoparticulate cephalosporin particles, i.e. not tablets, of less than 2000 nm that may be compressed into mini-tablets of undefined size that in turn may be blended into capsules.

Depending on the size, small-sized tablets are also referred to as mini-tablets. In the context of the present invention the term “mini-tablet” refers to a particle with a diameter of from 1-5 mm, preferably of from 1.5-4 mm, more preferably of from 1.7-3 mm.

In WO 2008/031782 mini-tablets are disclosed that comprise Factor Xa inhibitors. As shown in B. I. Eriksson et al. (Circulation (2006) 114, 2374-2381), such inhibitors have a so-called flat dose response across a wide dose range and do not require dose adjustments or patient monitoring. The above, combined with the fact that Factor Xa inhibitors are administered in relatively low amounts (i.e. approximately 10 mg), make them obvious targets for formulation in mini-tablets. In contrast, the treatment of bacterial infections with antibiotics such as β-lactam antibiotics, optionally in concert with β-lactamase inhibitors, requires careful administration. One reason is the occurrence of undesired bacterial resistance against antibiotics that may occur upon uncontrolled use of antibiotics. Today this bacterial resistance is one of the major threats in public health. Consequently, in combination with the fact that amounts of antibiotics to be administered are often relatively high, formulation of antibiotics in mini-tablets is not an obvious choice.

And even if formulation in a mini-tablet is considered, still the problem that arises when two different drugs, such as an antibiotic and a β-lactamase inhibitor, are to be formulated in a single granulate, tablet or mini-tablet, remains. This brings with it that an approach must be chosen that is a compromise of conditions that are best for each individual component. For example, formulation and storage of the β-lactamase inhibitor clavulanic acid dictates operation under strict (anhydrous) conditions whereas formulation of the antibiotic amoxicillin requires less stringent (and technical complex and expensive) conditions. Moreover, the regular form of amoxicillin used in antibiotic preparations is one which contains crystal water. Hence, formulating such combination drugs in a single operation and/or in a single tablet either requires technical features that need not be used for one constituent or avoids such conditions thereby creating a situation that is detrimental to the other. Similarly, the very existence of one drug in the same tablet as the other brings with it unwanted possibilities for detrimental interactions.

In a first aspect of the invention, disclosed is a composition comprising a first granule comprising an antibiotic and a second granule comprising a β-lactamase inhibitor, characterized in that said first granule and/or said second granule is a mini-tablet. For example, said composition comprises 2-150 mini-tablets, 5 to 100 mini-tablets, or 10 to 50 mini-tablets. Suitably, the granules in accordance with the invention are contained in a sachet for oral administration. In the context of the present invention the term “sachet” is to be interpreted as a small disposable bag or pouch used to contain a plurality of granules. Typically a sachet contains a single dose of the drug mixture, and the content of the sachet is taken by the patient at once. Sachets are made according to methods and using materials known to the skilled person such as polyethylene, polypropylene, polyethylene terephthalate and the like. Sachets can also be laminated with an aluminum layer. In a preferred embodiment a sachet is air-tight and/or light-tight. The sachet may or may not be filled with an inert gas or its interior may be under reduced pressure.

In a first embodiment of the present invention the diameter of the first and second granules is from 1 to 5 mm. The term “diameter” is to be interpreted as follows. For a circle the diameter is any straight line segment that passes through the center of the circle and whose endpoints are on the circle. The diameters are the longest chords of the circle. For a convex shape in the plane, the diameter is defined to be the largest distance that can be formed between two opposite parallel lines tangent to its boundary, and the width is defined to be the smallest of such distances. The same applies for shapes that are ‘circle-like’ or ‘convex-like’.

Suitably, the first and second granules have a diameter of less than 5 mm, 4.5 mm or less, or less than 4.5 mm, for example 0.2 to 4.5 mm, 0.4 to 4.5 mm, 1 to 4.5 mm, 2 to 5 mm, 2 to 4.5 mm, 2 to 4 mm, 2 to 3.5 mm, 2.5 to 5 mm, 2.5 to 4.5 mm, 2.5 to 4 mm, 2.5 to 3.5 mm, 3 to 5 mm, 3 to 4.5 mm, 3 to 4 mm, 3 to 3.5 mm, 3.1 to 3.3 mm or 3.2 mm. Suitably, the mini-tablets have a height (also referred to as thickness) of 6 mm or less, 4.5 mm or less, for example 0.2 to 4.5 mm, 0.5 to 4.5 mm, 1 to 4.5 mm, 2 to 5 mm, 2 to 4.5 mm, 2 to 4 mm, 2 to 3.5 mm, 2 to 3 mm, 2.4 to 2.6 mm or 2.5 mm. The first and second granules may have any shape convenient to the skilled person e.g. spherical or cylindrical. In one embodiment of the invention, the first and second granules are round and convex (known in the art as “round standard convex”). For example, the first and second granules may have the dimensions 3.2 diameter by 2.5 mm height.

In one embodiment, the first granule of the present invention suitably comprises from 50 to 99% of an antibiotic such as a β-lactam antibiotic and the like, based on the total weight of the first granule (unless otherwise stated, % compositions herein are based on the total weight of the first granule, including any film coating but excluding any sachet containing a plurality of granules). Examples of β-lactam antibiotics are aminopenicillins, first-generation cephalosporins, second-generation cephalosporins and third-generation cephalosporins. Examples of aminopenicillins suitable in the present invention are amoxicillin and ampicillin. Examples of first-generation cephalosporins are cefadroxil, cephalexin and cephradine. Examples of second-generation cephalosporins are cefaclor, cefprozil and cefuroxime axetil. Examples of third-generation cephalosporins are cefdinir, cefixime, cefpodoxime and ceftibuten.

In another embodiment, the second granule of the present invention suitably comprises from 10 to 99% of a β-lactamase inhibitor such as clavulanic acid, sulbactam or tazobactam.

In yet another preferred embodiment both the first and the second granule are a mini-tablet. The mini-tablet of the present invention may contain other substances known as excipients, which have specific functions. An excipient is defined as an inactive ingredient or any component other than the active ingredient added intentionally to the medicinal formulation or everything in the formulation except the active drug. Excipients are also called additives, pharmaceutical ingredients, or inactive pharmaceutical ingredients. The reasons for selecting and adding these excipients in formulations are to alter the physicochemical properties such as solubility, stability, color and the like. The bioavailability of drugs can also be varied by excipients.

Disintegrants are agents that may be added to the mini-tablet (and some encapsulated) formulations to promote the breakup of the tablet (and capsule “slugs’) into smaller fragments in an aqueous environment thereby increasing the available surface area and promoting a more rapid release of the drug substance. Starch is a well know disintegrant. In addition to starch, the following are some of the disintegrants which may be used. Pre-gelatinized Starch (Starch 1500) is a directly compressible form of starch consisting of intact and partially hydrolyzed ruptured starch grains. Pre-gelatinized starch has multiple uses in formulations as a binder, filler and disintegrant. As a disintegrant, its effective use concentration is between 5-10%. Microcrystalline cellulose (avicel) may be added because of its excellent flow and binding properties. It is also an effective mini-tablet disintegrant when used in a concentration of between 10-20%. Preferably, in addition to the normal disintegrants super disintegrants may be used to improve disintegration. Because of the increased demands for faster disintegration requirements, there are now available, a new generation of “Super Disintegrants” in addition to the disintegrants discussed earlier. Three major groups of compounds have been developed which swell to many times their original size when placed in water while producing minimal viscosity effects:
Modified starches such as sodium carboxymethyl starch (Chemically treated potato starch) such as sodium starch glycolate (Explotab, Primogel)
Cross-linked polyvinylpyrrolidone such as crosspovidone (Polyplasdone XL, Kollidon CL)
Modified Cellulose (internally cross-linked form of sodium carboxymethyl cellulose) such as Ac-Di-Sol (Accelerates Dissolution)

Flowing agents may be added as these help products to smoothly flow through the manufacturing equipment. Magnesium stearate is often advantageously used to help the finished products be released from the die and prevent the product sticking to the machinery. Silicon Dioxide is used in hydrophobic products that tend to clump together; this helps them enter the machinery correctly. Preferably silicon dioxide (Aerosil) is used.

Binding agents may be used in order to cause adhesion of powder particles in tablet granulations such as acacia, alginic acid, sodium-carboxymethylcellulose, compressible sugar, ethylcellulose gelatin, liquid glucose, methylcellulose, povidone, pre-gelatinized starch. Binding agents are often the same as the disintegrating agents. In case much binding agent is used a mixture of a simple, cheap binding agent like MCC (micro crystalline cellulose), di-calcium phosphate or lactose may be used in combination with a (super) disintegrant.

The mini-tablet of the present invention may be uncoated or coated with one or more layers of coating. Suitably, the mini-tablet is enteric coated. The enteric coating may comprise a pH dependent polymer, for example a copolymer of the methacrylic acid and methacrylic acid ester such as a methacrylic acid copolymer, for example Eudragit e.g. Eudragit L30D55 which has a disintegration above pH 5.5. Other Eudragits include: Eudragit L100-55 (disintegration above pH 5.5), Eudragit L100 (disintegration above pH 6.0) and Eudragit S100 (disintegration above pH 7.0). Suitably, the enteric coating comprises from 5 to 10% based on the total weight of the composition (dry polymer weight), suitably 6-8%. The enteric coating can be produced by spraying the enteric polymer on top of the above-described core mini-tablet.

Suitably, the enteric coating further comprises a plasticizer. Suitably, the mini-tablet of the present invention further comprises a plasticizer to aid in film formation during the film coating process, such as acetyl triethyl citrate or triethyl citrate, for example triethyl citrate (Citroflex). Suitably, the enteric coating further comprises a glidant. Suitably, the mini-tablet of the present invention further comprises a glidant to eliminate sticking during the film coating process such as talc, kaolin, or glycerol monostearate, for example glycerol monostearate (Imwitor 900K). Suitably, the enteric coating further comprises a surfactant to provide homogeneous film mixtures, such as sodium lauryl sulphate, polyethylene glycol, or polysorbate, for example Polysorbate 80 (Crillet 4HP). The mini-tablet may, if desired, further include one or more pharmaceutically acceptable excipients. All such excipients must be “pharmaceutically acceptable” in the sense of being compatible with the other ingredients of the pharmaceutical composition and not injurious to the patient. Pharmaceutically acceptable excipients may include colors, flavors such as menthol, sweeteners such as mannitol, preservatives, stabilizers, antioxidants and any other excipients known to those skilled in the art.

Coating agents may be applied for several reasons like creation of a moisture barrier to increase the stability of the active pharmaceutical ingredient, to ensure ease of administration (slippery coatings), to ensure proper taste masking or to color the tablets to change the appearance of the tablet. Typical coating types are known as (aqueous) film coatings, sugar coatings, enteric coatings.

Sweeteners and flavors may be added to help improve taste of the mini-tablet. A range of sweeteners may be used such as aspartame, dextrose monohydrate, erythitol, mannitol, maltodextrines and the like. Likewise a range of flavors and flavoring systems may be added such as strawberry, vanilla, orange and the like.

The mini-tablets of the present invention may comprise matrix polymers. Suitable matrix polymers include hydrophilic water soluble polymers, for example high molecular weight polymers (i.e. 100,000 to 800,000 Daltons), such as hydroxypropyl methylcellulose polymers (hypromellose). The mini-tablet may comprise one or more fillers, a suitable example of which is microcrystalline cellulose. In one embodiment of the invention, the filler is microcrystalline cellulose e.g. Avicel PH101. Avicel PH101 is microcrystalline cellulose with an average particle size of 50 μm. The mini-tablet may further comprise a glidant. Suitable glidants include colloidal silicon dioxide and talc. In one embodiment of the invention, the flow enhancer is colloidal silicon dioxide, for example Cab-O-Sil. Suitably, the mini-tablet further comprises a lubricant. Suitable lubricants include stearic acid, and stearic acid salts, for example magnesium stearate. In one embodiment of the invention, the lubricant is magnesium stearate.

In one embodiment of the invention, the total weight of the mini-tablet is from 5 to 200 mg, preferably from 6 to 150 mg and more preferably from 7 to 100 mg. For example, a 7.5 mg mini-tablet may contain 6.3±0.2 mg of amoxicillin trihydrate and diluents and/or fillers and/or binders and/or lubricants and/or disintegrating agents and/or coating agents.

Mini-tablets have the following advantages such as ease of use by sprinkling them over food, ease of use by putting them in drinks and quickly dispersion is preferred in this case (done by the addition of a dispersant). When packaged in a sachet, mini-tablets can also be taken as a single dose in the mouth, preferably with a proper taste masking agent present, or a taste masking coating around the mini-tablets. Mini-tablets can be put on a spoon and by the addition of water form a dispersion similar to the ones obtained through pharmacies. Mini-tablets can be taken one by one or in small portions. Conventional (i.e. large) tablets are often crushed into smaller ones. This is not necessary in case of using mini-tablets. Crushing often leads to loss of material or damage of the protective outer coating (if applied to tablets) and cause loss of activity. Finally, mini-tablets are recognized as child friendly formulations, since they facilitate administration of the active pharmaceutical ingredient. The same holds true for the use of mini-tablets for elderly. For these applications, preferred sizes of mini-tablets are from 1.5-3 mm.

In another embodiment the compositions of the present invention comprise mini-tablets that comprise a β-lactam antibiotic, preferred examples of which are amoxicillin, ampicillin, cefoperazone, piperacillin, ticarcillin and pharmaceutically acceptable salts thereof and further comprise mini-tablets that comprise a β-lactamase inhibitor, preferred examples of which are clavulanic acid, sulbactam, tazobactam and pharmaceutically acceptable salts thereof. The latter embodiment is advantageous in cases where either the β-lactam antibiotic or the β-lactamase inhibitor or both require different additives and/or must be formulated in one another's absence, e.g. for stability reasons. A further advantage is easier production resulting from higher flexibility in mixing appropriate amounts of the respective mini-tablets. The most preferred combinations of β-lactam antibiotic and β-lactamase inhibitor are amoxicillin plus clavulanic acid, ampicillin plus sulbactam, cefoperazone plus sulbactam, piperacillin plus tazobactam and ticarcillin plus clavulanic acid. For example, a composition can contain, next to the mini-tablets with amounts of amoxicillin trihydrate mentioned above, also granulates comprising clavulanic acid such that ratio's amoxicillin trihydrate to clavulanic acid of 1:1, 5:1, 6:1, 10:1, 11:1, 15:1, 16:1 or 20:1 are obtained.

In a second aspect of the present invention there is provided a process for preparing a mini-tablet as present in the composition of the first aspect. Mini-tablets are tablets formed by using a tablet press or a roller compaction device with special shaped pressure roles. The product from both is a compacted powder, a tablet, in the size range of 0.9-5 mm diameter (this is possible to make size-wise) and a height of 1-10 mm. Preferred is a size between 1.5-3 mm diameter and 1-6 mm height). In one embodiment the preferred mini-tablets are 2±0.2 mm in diameter and 2.2±0.2 mm in height. The preferred process is tableting as this results in more accurate uniformity in size of the resulting mini-tablets. The mini-tablet is suitably prepared by, in one or more stages, combining the components, granulating, drying, milling, and compressing the mixture into tablets. In one embodiment, the mini-tablet is prepared using a wet granulation method, such as are well known in the art. For example, amoxicillin, a filler, a polymer and sufficient amounts of a granulating fluid such as water are combined, granulated, dried and milled to form granules. The dried granules are milled to achieve a suitable particle size, for example a D₅₀ (50^{th} percentile of the cumulative particle size distribution) between 50 to 300 microns (μm), for example 100-300 microns or 100-200 microns. The granules are then combined with the remaining components, for example using a high shear mixing process, and the mixture is compressed into the mini-tablet. The mini-tablet is then optionally coated with an enteric coating composition.

In one embodiment in a process for preparing a mini-tablet as present in the composition of the first aspect, amoxicillin or clavulanic acid (a 50:50 w/w % with Avicel 101) are mixed together in the appropriate amounts and subsequently mixed with the other excipients excluding the Mg-stearate. Mixing may be done in a standard way known in the field such as by means of tumbler mixing or (high) shear mixing. When mixing is done, the final amount of Mg-stearate is added. This is preferably done in the second stage to make sure the Mg-stearate, or any alternate stearic acid derivative, is working properly as a glidant during tableting. The resulting mixture may be fed into a tablet press like the Fette RoTab T, M or MinTab T machine supplied by Kg-Pharma. In one embodiment the mini-tablets are made using a multi tip tool. A multiple tip tool is a punch body that houses more than one tip per punch therefore allowing for the production of more than one tablet per station on the press turret. This means that it is possible to produce several times the output of a press running with standard single tip punches. Each punch is fitted with the required amount of tips. The tip number changes with the size of the mini-tablets. For example, a 2 mm tip of category B has 16 tips per punch, the D-type tool has 35 tips. These punches are supplied by many companies. Suitable examples are the Fette punches, like Fette 441. The powder is tableted to the required strength, which may be set in the tablet press and measured afterwards. Suitable equipment for testing may be a DIYTrade YD-1 Tablet hardness tester, A ERWEKA TBH-28 hardness tester or a Casburt C53 Tablet hardness tester. Also the friability may be measured, for example using a Key International Inc Ft-400 Friability tester or an ERWEKA TA-UZ friability tester.

Disintegration is tested according to the European and Japanese Pharmacopoeia. The apparatus consist of a basket-rack assembly which is immersed in a temperature controlled liquid, in this case tap water. The basket is raised and lowered in the liquid at a constant frequency between 29 and 32 cycles per minute over a distance between 53 and 57 mm. The volume of the fluid in the vessel is such that at the highest point of the upward stroke the wired mesh of the basket remains at least 15 mm below the surface of the liquid and descends to not more than 25 mm from the bottom of the vessel on the downward stroke. In the basket a rack is present consisting of six open ended tubes in which the tablets are placed when measuring the disintegration rates. The mesh should be such that the tablets cannot fall out of the tube bottom during the movement of the device. The disintegration may be measured using tap water of 37±2° C. The time to full disintegration is monitored by recording the moment that the tablet has fully disappeared from the tube. The insoluble parts of the formulation will fall through the mesh below the tube in which the tablets were placed.

In another embodiment the obtained mini-tablets are coated with a coating solution in a tablet coater like the Vector LDCS-Hi-Coater using an Eudragit L100-55 solution.

In a third aspect the present invention provides a pharmaceutical composition for the manufacture of a medicament for the treatment of a patient suffering from an antibacterial infection.

In a fourth aspect the present invention provides a pharmaceutical composition for use in the treatment of a bacterial infection. In one embodiment said use is particularly suitable in children. Since the mini-tablets are made using a tableting or compaction process, processes well known in the field and proven effective for the production of active pharmaceutical ingredient containing formulations, the accurate dosing of an active pharmaceutical ingredient by using mini-tablets is assured. Typically, mini-tablets are dosed in certain amounts, thus ensuring the administration of the right dose to the patient. In the context of the present invention this preferably is 10-60 mini-tablets per dose, more preferably 20-50 mini-tablets per dose, most preferably 30-40 mini-tablets per dose. The above is important to mention since medical practitioners emphasize the correct dose to be most important. Parents are more concerned with the fact of getting the active pharmaceutical ingredient administered without spitting out, vomiting or rejection the next time the dose needs to be taken. Also the smell and difficulty of swallowing are mentioned in many surveys. Parents and staff of care centers report that they often crush or break conventional (i.e. large) tablets for better take up or mix the medicine with food or drinks. For all of these issues the mini-tablets have a great advantage. Hence, in one embodiment the mini-tablet of the present invention is applied on or in a foodstuff. In the context of the present invention the term “foodstuff” is to be understood as an instance of material which may be used as food. Such instance may be solid or liquid (i.e. a beverage). Examples of suitable foodstuffs are bread in any form (i.e. baked, fried and the like), butter, condiments, dairy products such as cheese, milk, yoghurt and the like, jam, meat in any form (i.e. baked, boiled, fried and the like), milk, peanut butter, potatoes in any form (i.e. baked, boiled, fried and the like), rice in any form (i.e. baked, boiled, fried and the like), soy-products, sweets, toast, vegetables in any form (i.e. baked, boiled, fried and the like).

In a fifth aspect the present invention provides a plurality of pharmaceutical compositions arranged in a pharmaceutical package, conveniently with instructions for use.

The following examples illustrate the present invention but should not be construed as limiting the scope.

### EXAMPLES

### Example 1

### Preparation of Mini-Tablets Comprising Amoxicillin

TABLE 1
Mini-tablets made based on amoxicillin only (Series 1-4) or a 125/31.25
mg/mg amoxicillin trihydrate/clavulanic acid content (Series 5-8)

| | Orange flavor | Banana flavor | Sugar | Blank |
|---|---|---|---|---|
| | Series 1 | Series 2 | Series 3 | Series 4 |
| Ingredients | % | % | % | % |
| Amoxicillin•3H₂0 | 84 | 84 | 84 | 84 |
| Avicel PH112 | 9.45 | — | 8 | 9 |
| Mannitol | — | 9.45 | — | — |
| Saccharin | — | — | 2 | 1.5 |
| Ac-di-sol | 3 | 3 | 3 | 3 |
| Aspartame | 0.5 | 0.5 | 0.5 | — |
| Aerosil | 1 | 1 | 1 | 1 |
| Orange flavor | 0.25 | — | — | — |
| Banana flavor | — | 0.25 | — | — |
| Mg Stearate | 1.5 | 1.5 | 1.5 | 1.5 |
| Total | 100 | 100 | 100 | 100 |
| | Orange flavor | Banana flavor | Sugar | Blank |
| | Series 5 | Series 6 | Series 7 | Series 8 |
| Ingredients | % | % | % | % |
| Amoxicillin dry | 60.2 | 60.2 | 60.2 | 60.2 |
| Potassium | 32.3 | 32.3 | 32.3 | 32.3 |
| clavulanate (as | | | | |
| potassium | | | | |
| clavulanate: | | | | |
| avicel 1:1) | | | | |
| Ac-di-sol | 4.0 | 4.0 | 2.6 | 3.6 |
| Aerosil | 1 | 1 | 1 | 1 |
| Aspartame | 0.5 | 0.5 | 0.5 | — |
| Saccharin | 0 | 0 | 2 | 1.5 |
| Orange flavor | 0.27 | — | — | 1 |
| Banana flavor | — | 0.25 | — | — |
| Mg Stearate | 1.5 | 1.5 | 1.5 | 1.5 |
| Total | 100 | 100 | 100 | 100 |

The amoxicillin only (series 1-4 in Table 1) or the amoxicillin and the clavulanic acid (a 50:50 w/w % with Avicel 101; series 5-8 in Table 1) were mixed together in the appropriate amounts (see Table 1) and mixed with the other excipients excluding Mg-stearate using a tumbler mixer. After mixing for 20 min, the amount of Mg-stearate as indicated in Table 1 was added and mixing was continued for another 5 min in the tumbler mixer. Subsequently, the mixture was fed into a the Fette RoTab T tablet press (supplied by Kg-Pharma).

### Example 2

### Preparation of Mini-Tablets Comprising Ampicillin, Cefaclor, Cefadroxil, Cefdinir, Cefixime, Cefpodoxime, Cefprozil, Ceftibuten, Cefuroxime Axetil, Cephalexin or Cephradine

In analogy with Example 1, Series 1-4, mini-tablets can be prepared by replacing the 84% amoxicillin trihydrate with 84±3% of ampicillin or cefaclor or cefadroxil or cefdinir or cefixime or cefpodoxime or cefprozil or ceftibuten or cefuroxime axetil or cephalexin or cephradine.

### Example 3

### Stability Data

The 8 mini-tablets prepared in Example 1 and an amoxicillin reference sample were analyzed for stability. The results are given in Table 2. Stability data for clavulanic acid in mini-tablets compared to a clavulanic acid reference sample were also analyzed for stability. The results are given in Table 3.

TABLE 2
Stability of mini-tablets made based on amoxicillin
only (Series 1-4) or a 125/31.25 mg/mg amoxicillin
trihydrate/clavulanic acid content (Series 5-8) compared to
an amoxicillin reference sample as a function of time.

| Weeks | 0 | 1 | 4 | 10 | 18 | 49 |
|---|---|---|---|---|---|---|
| Series 1 | 100.0 | 100.0 | 100.2 | 99.9 | 100.2 | 97.7 |
| Series 2 | 100.0 | 100.0 | 98.8 | 100.5 | 99.3 | 99.7 |
| Series 3 | 100.0 | 100.0 | 101.0 | 99.1 | 97.7 | 99.4 |
| Series 4 | 100.0 | 100.0 | 98.8 | 100.9 | 98.2 | 97.6 |
| Series 5 | 100.0 | 100.0 | 101.0 | 100.6 | 99.6 | 99.2 |
| Series 6 | 100.0 | 100.0 | 100.0 | 101.8 | 100.0 | 98.1 |
| Series 7 | 100.0 | 100.0 | 100.0 | 98.3 | 99.3 | 96.2 |
| Series 8 | 100.0 | 100.0 | 100.3 | 99.0 | 98.5 | 94.8 |
| Amoxicillin | 100.0 | 100.0 | | | | 97.8 |
| reference | | | | | | |

TABLE 3
Stability of mini-tablets containing clavulanic acid compared
to a clavulanic acid reference sample as a function of time.

| Weeks | 0 | 1 | 4 | 8 | 18 | 26 |
|---|---|---|---|---|---|---|
| Series 5 | 100.0 | 100.0 | 95.7 | 91.6 | 88.2 | |
| Series 6 | 100.0 | 100.0 | 95.1 | 91.0 | 87.4 | |
| Series 7 | 100.0 | 100.0 | 98.0 | 93.8 | 89.5 | |
| Series 8 | 100.0 | 100.0 | 99.1 | 94.9 | 93.3 | |
| Clavulanic acid reference | 100.0 | 86.4 | | | | 88.4 |

### Example 4

### Preparation of Mini-Tablets Comprising Amoxicillin and Direct Compression (DC Amoxicillin

Formulation 2DC (Table 4) was made with compacted amoxicillin, the amoxicillin was sieved before and the fraction <425 μm was used in the formulation. The other formulations were made with amoxicillin powder. The mini-tablets have a diameter of 2 mm. Formulation 4 is a placebo mixture without active ingredient.

TABLE 4
First series of formulation mixtures of the tablets (amoxicillin only)

| Ingredients | | | | | | |
|---|---|---|---|---|---|---|
| (%) | Formulation | 1 | 2 | 2DC | 3 | 4 |
| Active | Amoxicillin | 84.2 | 71.9 | 71.9 | 68.6 | — |
| Disintegrants | Ac-Di-Sol | — | — | — | 4.76 | — |
| Sugars | Saccharin | 1.98 | 1.69 | 1.69 | 1.61 | 2.00 |
| | Sorbitol | — | — | — | — | 85.1 |
| Other | Avicel pH112 | 11.2 | 25.0 | 25.0 | 23.8 | 11.4 |
| excipients | Aerosil | 0.59 | 0.51 | 0.51 | 0.48 | 0.60 |
| | Mg Stearate | 1.98 | 0.85 | 0.85 | 0.81 | 1.00 |

These tablets were used to measure the weight, size, hardness and disintegration time of the mini-tablets (Table 5).

TABLE 5
First series of formulation mixtures of the tablets (amoxicillin only)

| | Weight | Height | Hardness | Disintegration |
|---|---|---|---|---|
| Formulation | (mg) | (mm) | (Kp = N/10) | (sec) |
| 1 | 7.51 | 2.20 | 0.88 | 22 |
| 2 | 7.35 | 2.10 | 0.63 | 20 |
| 2DC | 8.02 | 2.45 | 0.86 | 10 |
| 3 | 7.29 | 2.11 | 1.15 | 10 |
| 4 | 7.11 | 2.00 | 1.72 | 300 |

The disintegration of the mini-tablets is fast for formulations 1 and 2 (20-22 s) and very fast for the 2DC (amoxicillin direct compacted material) and formulation 3. Formulation 4 shows placebo mini-tablets and shows that a very hard tablet can have a very high disintegration time.

### Example 5

### Optimized Preparation of Mini-Tablets Comprising Amoxicillin

Mini-tablets similar to the ones described in Example 1 were prepared and applied in the following dosage forms:
One dosage for a child is 125 mg Amoxicillin and 32.5 mg K-Clavulanate.
The amount of mini-tablets for formulation 1-4=24 mini-tablets per dosage
And for the mixture formulations 5-8=34 mini-tablets per dosage

### Example 6

### Hardness, Friability and Disintegration of Mini-Tablets

The 8 mini-tablets prepared in Example 1 were analyzed for hardness, friability and disintegration. Hardness was tested using an ERWEKA TBH-28 Tablet hardness tester. Friability was measured using an ERWEKA TA-UZ Friability tester. Tablet disintegration was measured using an ERWEKA Disintegration tester ZT3 with a thermostatic bath. Disintegration was measured as outlined above.

The results are given in Table 6.

TABLE 6
Properties of the mini-tablets made in Example 1

| | | | | | Disinte- |
|---|---|---|---|---|---|
| | Weight | Height | Hardness | Friability (% | gration |
| Formulation | (mg) | (mm) | (Kp = N/10) | weight loss) | (sec) |
| Series 1 | 7.53 | 2.2 | 0.62 | 0.07 | 10 |
| Series 2 | 7.51 | 2.2 | 0.54 | 0.13 | 10 |
| Series 3 | 7.52 | 2.2 | 0.70 | 0.13 | 15 |
| Series 4 | 7.47 | 2.2 | 0.57 | 0.00 | 10 |
| Series 5 | 7.67 | 2.2 | 0.26 | 0.00 | 45 |
| Series 6 | 7.64 | 2.2 | 0.05 | 0.00 | 30 |
| Series 7 | 7.59 | 2.2 | 0.08 | 1.75 | 45 |
| Series 8 | 7.59 | 2.2 | 0.00 | 0.20 | 12 |

1. A composition comprising a first granule comprising an antibiotic and a second granule comprising a β-lactamase inhibitor, characterized in that said first granule and/or said second granule is a mini-tablet.

2. Composition according to claim 1 wherein the diameters of said first and second granules independently are from 1 to 5 mm.

3. Composition according to claim 1 wherein the heights of said first and second granules independently are from 1 to 6 mm.

4. Composition according to claim 1 wherein said antibiotic is a β-lactam antibiotic.

5. Composition according to claim 4 wherein said β-lactam antibiotic is amoxicillin.

6. Composition according to claim 1 wherein said β-lactamase inhibitor is clavulanic acid.

7. Composition according to claim 1 which is contained in a sachet.

8. Composition according to claim 7 wherein said sachet comprises from 5 to 100 of said first granules and from 5 to 100 of said second granules.

9. Method for the production of a mini-tablet comprising the steps of mixing active ingredients with excipients followed by tableting the resulting mixture.

10. Method according to claim 9 wherein said mixing is carried out in two subsequent stages whereby magnesium stearate is added in the second stage.

11. Composition according to claim 1 for use as an antibacterial agent.

12. Composition according to claim 11 which is applied on or in a foodstuff.
